(19) United States
(12) Patent Application Publication
(10) Pub. No.: US 2022/0202804 A1
(43) Pub. Date: Jun. 30, 2022

(54) COMPOSITIONS AND METHODS TO TREAT CANCER

(71) Applicant: Coherus Biosciences, Inc., Redwood City, CA (US)

(72) Inventor: Glenn D. Rosen, Los Altos, CA (US)

(21) Appl. No.: 17/613,012

(22) PCT Filed: May 26, 2020
(86) PCT No.: PCT/US2020/034480

§ 371 Date: Nov. 19, 2021

Related U.S. Application Data
(60) Provisional application No. 62/854,569, filed on May 30, 2019.

(51) Int. Cl. A61K 31/47; A61K 33/243; A61K 31/282; A61P 35/00

(52) U.S. Cl. CPC A61K 31/47; A61P 35/00; A61K 31/282; A61K 33/243

(57) ABSTRACT

Provided herein are methods and combination therapies useful for the treatment of cancer. In particular, provided herein are methods and combination therapies for treating cancer by administering a combination therapy comprising (a) the compound of Formula (I), or a pharmaceutically acceptable salt thereof, and (b) at least one DNA damaging agent, or a pharmaceutically acceptable salt thereof. Also provided are pharmaceutical compositions and pharmaceutical combinations comprising the compound of Formula (I), or a pharmaceutically acceptable salt thereof, and at least one DNA damaging agent, or a pharmaceutically acceptable salt thereof.

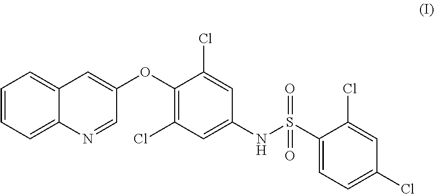 [SMILES: O=S(=O)(Nc1cc(Cl)c(Oc2cnc3ccccc3c2)c(Cl)c1)c1ccc(Cl)cc1Cl]

### CROSS-REFERENCE TO RELATED APPLICATION

This application claims priority to U.S. Provisional Patent Application Ser. No. 62/854,569, filed May 30, 2019, the entire contents of which are herein incorporated by reference.

### TECHNICAL FIELD

The present disclosure relates to methods and combination therapies useful for the treatment of cancer. In particular, this disclosure relates to methods and combination therapies for treating cancer by administering a combination therapy comprising a PPARγ inhibitor that is the compound of Formula (I), or a pharmaceutically acceptable salt thereof, and at least one DNA damaging agent, or a pharmaceutically acceptable salt thereof.

### BACKGROUND

Cancer is characterized by uncontrolled cell growth and proliferation, and dysregulation of cell death pathways. Peroxisome proliferator-activated receptors (PPARs) are ligand-activated transcription factors that belong to the nuclear hormone receptor superfamily that bind to a direct repeat of two hexanucleotides, spaced by one or two nucleotides, as heterodimers with the retinoid X receptor (RXR), and activate many target genes involved in various pathways that can impact tumor development and progression. Hsu, et al., J. Biol. Chem., 273(43), pp. 27988-27997 (1998).

The average 5-year survival rates for common pediatric and adult cancers in North America are 65-95% and 14-56% respectively, which remain quite low. See Altekruse, et al., SEER Cancer Statistics Review, 1975-2007, National Cancer Institute, Bethesda, Md.; and Gatta, et al., Eur. J. Cancer, 45, pp. 992-1005 (2009). While age-standard mortality rates for all cancer types have decreased, only a small percentage of this change is due to new pharmacological agents, largely due to the expense and extensive trials required to come to market. See Garattini, et al., Ann. Oncol., 14, pp. 813-816 (2003); and DiMasi, et al., J. Health Econ., 22, pp. 151-185 (2003). Consequently, approaches that do not rely solely on a single agent's traditional cytotoxicity profile are required in order to provide a more targeted, efficient, and enhanced form of cancer therapy. See Mokhtari, et al., Oncotarget, Vol. 8, No. 23, pp. 38022-38043(2017). For instance, combinations of therapeutic agents that will be efficacious in treating cancer.

### SUMMARY

Provided herein in some embodiments is a method of treating cancer in a subject in need thereof comprising administering to the subject
(a) the compound of Formula (I),

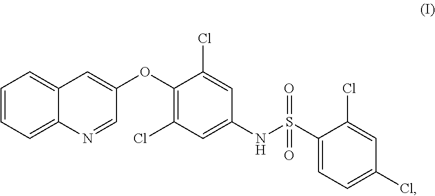 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) at least one DNA damaging agent, or a pharmaceutically acceptable salt thereof,
   wherein the amounts of (a) and (b) together are effective in treating cancer.

Provided herein in some embodiments is a method of treating a subject, the method comprising:

selecting a subject having cancer; and

administering
(a) the compound of Formula (I),

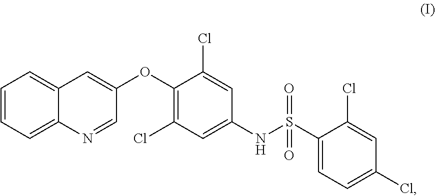 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) at least one DNA damaging agent, or a pharmaceutically acceptable salt thereof, to the selected subject,
   wherein the amounts of (a) and (b) together are effective in treating cancer.

Provided herein in some embodiments is a method of treating a subject, the method comprising:

identifying a subject having cancer; and

administering
(a) the compound of Formula (I),

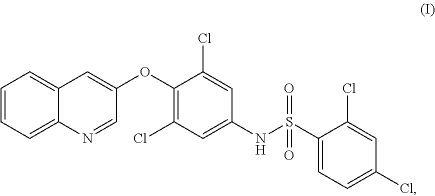 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) at least one DNA damaging agent, or a pharmaceutically acceptable salt thereof, to the selected subject,
   wherein the amounts of (a) and (b) together are effective in treating cancer.

Provided herein in some embodiments is a method of treating cancer in a subject in need thereof comprising administering to the subject
(a) a therapeutically effective amount of the compound of Formula (I),

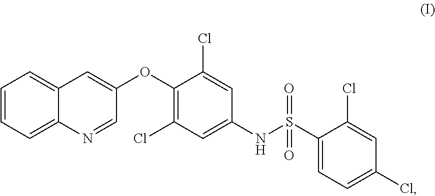 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) a therapeutically effective amount of at least one DNA damaging agent, or a pharmaceutically acceptable salt thereof.

Provided herein in some embodiments is a method of treating a subject, the method comprising:

selecting a subject having cancer; and

administering
(a) a therapeutically effective amount of the compound of Formula (I),

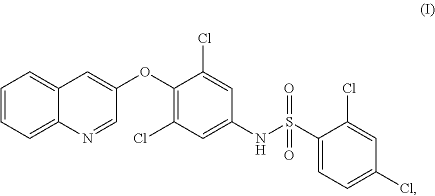 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) a therapeutically effective amount of at least one DNA damaging agent, or a pharmaceutically acceptable salt thereof, to the selected subject.

Provided herein in some embodiments is a method of inhibiting cancer cell proliferation in a subject in need thereof, the method comprising contacting the cancer cell with
(a) a therapeutically effective amount of the compound of Formula (I),

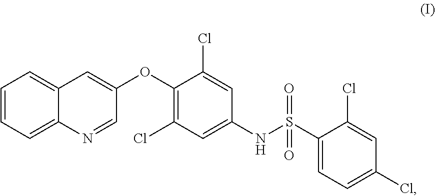 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) a therapeutically effective amount of at least one DNA damaging agent, or a pharmaceutically acceptable salt thereof.

Provided herein in some embodiments is a method of treating cancer in a subject in need thereof consisting essentially of administering to the subject
(a) the compound of Formula (I),

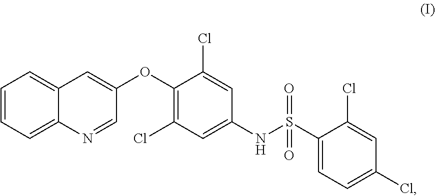 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and
(b) one DNA damaging agent, or a pharmaceutically acceptable salt thereof, selected from: cyclophosphamide, mechlorethamine, chlorambucil, melphalan, dacarbazine, temozolomide, cisplatin, carboplatin, oxaliplatin, nedaplatin, triplatin tetranitrate, phenanthriplatin, picoplatin, satraplatin, carmustine, lomustine, semustine, bendamustine, nimustine, ranimustine, uramustine, busulfan, chlormethine, fotemustine, ifosfamide, streptozocin, bleomycin, mitomycin, altretamine, irinotecan, topotecan, camptothecin, lamellarin, etoposide, teniposide, doxorubicin, daunorubicin, epirubicin, idarubicin, valrubicin, tafluposide mitoxantrone, and amsacrine; and pharmaceutically acceptable salts thereof;
wherein the amounts of (a) and (b) together are effective in treating cancer.

Provided herein in some embodiments is a pharmaceutical composition comprising
(a) the compound of Formula (I),

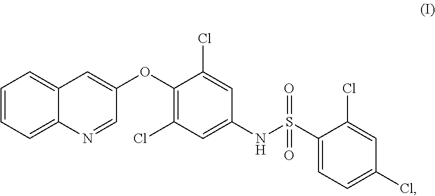 [SMILES: O=S(=O)(Nc1cc(Cl)c(Oc2cnc3ccccc3c2)c(Cl)c1)c1ccc(Cl)cc1Cl]

or a pharmaceutically acceptable salt thereof,
(b) at least one DNA damaging agent, or a pharmaceutically acceptable salt thereof, and

one or more pharmaceutical excipients. In some embodiments, the amounts of (a) and (b) together are effective in treating cancer.

In some embodiments, the amount of (a) is a therapeutically effective amount and the amount of (b) is a therapeutically effective amount.

Provided herein in some embodiments is a pharmaceutical combination comprising
(a) the compound of Formula (I),

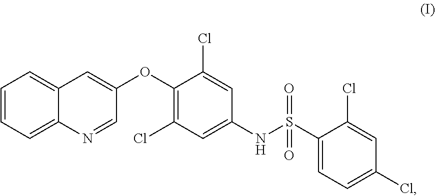 [SMILES: O=S(=O)(Nc1cc(Cl)c(Oc2cnc3ccccc3c2)c(Cl)c1)c1ccc(Cl)cc1Cl]

or a pharmaceutically acceptable salt thereof,
(b) at least one DNA damaging agent, or a pharmaceutically acceptable salt thereof, and

one or more pharmaceutical excipients, for concurrent or sequential administration during a period of time for use in the treatment of cancer. In some embodiments, the amounts of (a) and (b) together are effective in treating cancer.

Unless otherwise defined, all technical and scientific terms used herein have the same meaning as commonly understood by one of ordinary skill in the art. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present disclosure will be apparent from the following detailed description and figures, and from the claims.

### DETAILED DESCRIPTION

### Definitions

Reference to the term “about” has its usual meaning in the context of pharmaceutical compositions to allow for reasonable variations in amounts that can achieve the same effect and also refers herein to a value of plus or minus 10% of the provided value. For example, “about 20” means or includes amounts from 18 up to and including 22.

The term “administration” or “administering” refers to a method of giving a dosage of a compound or pharmaceutical composition to a vertebrate or invertebrate, including a mammal, a bird, a fish, or an amphibian. The preferred method of administration can vary depending on various factors, e.g., the components of the pharmaceutical composition, the site of the disease, and the severity of the disease, as understood by one skilled in the art.

The term “CHS-131” as used herein refers to a compound of Formula (I):

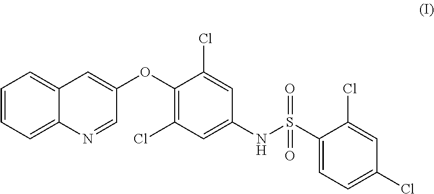 [SMILES: O=S(=O)(Nc1cc(Cl)c(Oc2cnc3ccccc3c2)c(Cl)c1)c1ccc(Cl)cc1Cl]

or a pharmaceutically acceptable salt thereof.
The compound of Formula (I) is a selective peroxisome proliferator-activated receptor (PPAR) γ modulator. The compound of Formula (I) is disclosed in, for example, U.S. Pat. Nos. 7,041,691; 6,200,995; 6,583,157; 6,653,332; and U.S. Publication Application No. 2016/0260398, the contents of each of which are incorporated by reference herein in their entireties.

The compound of Formula (I) can be prepared, for example, by the methods described in U.S. Pat. Nos. 6,583,157 or 6,200,995, each of which is incorporated by reference in its entirety herein. In some embodiments, different salts, e.g., besylate, tosylate HCl, or HBr salts, and/or polymorphs of the compound of Formula (I) are used within the methods and compositions described herein. Salts and polymorphs of the compound of Formula (I), such as those provided herein, can be prepared according to the methods described in U.S. Pat. Nos. 6,583,157 and 7,223,761, the contents of each of which are incorporated by reference in their entireties.

The term “DNA damaging agent” as used herein refers to a compound that causes damage to DNA. Such damage can include, for example, undesired covalent modification, undesired single and double strand breaks, and undesired covalent crosslinking with other biomolecules such as DNA and/or protein. DNA damaging agents include, but are not limited to, alkylating agents, DNA strand-breaking agents (including topoisomerase inhibitors), crosslinking agents. For example, DNA damaging agents include, but are not limited to: cyclophosphamide, mechlorethamine, chlorambucil, melphalan, dacarbazine, temozolomide, cisplatin, carboplatin, oxaliplatin, nedaplatin, triplatin tetranitrate, phenanthriplatin, picoplatin, satraplatin, carmustine, lomustine, semustine, bendamustine, nimustine, ranimustine, uramustine, busulfan, chlormethine, fotemustine, ifosfamide, streptozocin, bleomycin, mitomycin, altretamine, irinotecan, topotecan, camptothecin, lamellarin, etoposide, teniposide, doxorubicin, daunorubicin, epirubicin, idarubicin, valrubicin, tafluposide mitoxantrone, and amsacrine; and pharmaceutically acceptable salts thereof.

In some embodiments described herein, the compound of Formula I is a free base. In other embodiments, the compound of Formula I is a pharmaceutically acceptable salt, for example a hydrochloride salt. In some embodiments, the at least one DNA damaging agent is a free base. In other embodiments, the at least one DNA damaging agent is a pharmaceutically acceptable salt, for example, a hydrochloride salt.

As used herein, a “therapeutically effective amount” is an amount sufficient to achieve any one or more beneficial or desired results (the “therapeutic effect”) and can vary according to the nature and severity of the disease condition, and the potency of the compound. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include providing a therapeutic effect can include reducing the size of a tumor, inhibiting (e.g., slowing, to some extent, preferably stopping) cancer progression, inhibiting (e.g., slowing, to some extent, preferably stopping) cancer growth, inhibiting (e.g., slowing, to some extent, preferably stopping) cancer invasiveness, increasing patient survival, increased tumor cell death, inhibiting (e.g., slowing, to some extent, preferably stopping) a reduction in tumor cell death, and/or inhibiting (e.g., slowing, to some extent, preferably stopping) tumor metastasis. The skilled person understands that tumor progression in human subjects can be determined by a variety of methods. For example, the size of a tumor close to the skin can be measured by establishing the width and depth of the tumor with calipers, and then calculating the tumor volume. Less accessible tumors, such as lung and brain cancers can be measured by observation of the images obtained from Magnetic Resonance Imaging (MRI) scanning. Providing a therapeutic effect also includes prolonging survival of a subject beyond that expected in the absence of treatment and/or relieving to some extent (or preferably eliminating) one or more signs or symptoms associated with cancer. In some embodiments, treatment of a subject with a compound or combination as described herein prolongs survival beyond that expected in the absence of treatment by 1 or months, e.g., by 3 or more months, e.g., by 6 or more months, e.g., by 1 or more years, e.g., by 2 or more years, e.g., by 3 or more years, e.g., by 5 or more years, e.g., by 10 or more years. Providing a therapeutic effect also includes reducing the number of cancer cells. Providing a therapeutic effect also includes eliminating cancer cells. Providing a therapeutic effect also includes tumor mass reduction. Providing a therapeutic effect also includes causing a cancer to go into remission. A therapeutically effective amount can be administered in one or more administrations. As used herein, dosage of a therapeutically effective amount of a compound, or pharmaceutical composition thereof is an amount sufficient to accomplish prophylactic or therapeutic treatment either directly or indirectly. In reference to the treatment of cancer, a therapeutically effective amount may also refer to that amount which has the effect of (1) reducing the size of the tumor, (2) inhibiting (that is, slowing to some extent, preferably stopping) cancer metastasis emergence, (3) inhibiting to some extent (that is, slowing to some extent, preferably stopping) cancer growth or cancer invasiveness, and/or (4) relieving to some extent (or, preferably, eliminating) one or more signs or symptoms associated with the cancer. Therapeutic or pharmacological effectiveness of the doses and administration regimens may also be characterized as the ability to induce, enhance, maintain or prolong disease control and/or overall survival in subjects with these specific tumors, which may be measured as prolongation of the time before disease progression.

The term “synergy” or “synergistic” is used herein to mean that the effect of the combination of the two therapeutic agents of the combination therapy is greater than the sum of the effect of each agent when administered alone. A “synergistic amount” or “synergistically effective amount” is an amount of the combination of the two combination partners that results in a synergistic effect, as “synergistic” is defined herein. Determining a synergistic interaction between two combination partners, the optimum range for the effect and absolute dose ranges of each component for the effect may be definitively measured by administration of the combination partners over different w/w (weight per weight) ratio ranges and doses to subjects in need of treatment. However, the observation of synergy in in vitro models or in vivo models can be predictive of the effect in humans and other species and in vitro models or in vivo models exist, as described herein, to measure a synergistic effect and the results of such studies can also be used to predict the therapeutically effective amount and plasma concentration ratio ranges and the absolute doses and plasma concentrations required in humans and other species by the application of pharmacokinetic/pharmacodynamic methods. Exemplary synergistic effects includes, but are not limited to, enhanced therapeutic efficacy, decreased dosage at equal or increased level of efficacy, reduced or delayed development of drug resistance, and simultaneous enhancement or equal therapeutic actions (e.g., the same therapeutic effect as at least one of the therapeutic agents) and reduction of unwanted drug effects (e.g., side effects and adverse events) of at least one of the therapeutic agents. For example, a synergistic ratio of two therapeutic agents can be identified by determining a synergistic effect in, for example, an art-accepted in vivo model (e.g., an animal model) of cancer

As used herein, “subject” refers to any subject, particularly a mammalian subject, for whom diagnosis, prognosis, or therapy is desired, for example, a human.

As used herein, the term “contacting” refers to the bringing together of indicated moieties in an in vitro system or an in vivo system. For example, “contacting” a cancer cell with a compound provided herein includes contacting a sample containing the cell in a subject, as well as, for example, introducing a compound of Formula (I), or a pharmaceutically acceptable salt thereof, and at least one DNA damaging agent, or a pharmaceutically acceptable salt thereof, into a sample containing a cellular or purified preparation containing the cancer cell.

The terms “treatment regimen” and “dosing regimen” are used interchangeably to refer to the dose and timing of administration of each therapeutic agent in a combination as described herein.

The term “pharmaceutical combination”, as used herein, refers to a pharmaceutical treatment resulting from the mixing or combining of more than one active ingredient and includes both fixed and refers to non-fixed combinations of the active ingredients.

The term “combination therapy” as used herein refers to a dosing regimen of two different therapeutically active agents (i.e., the components or combination partners of the combination) (e.g., the compound of Formula (I), or a pharmaceutically acceptable salt thereof, and at least one DNA damaging agent), wherein the therapeutically active agents are administered together or separately in a manner prescribed by a medical care taker or according to a regulatory agency as defined herein. In some embodiments, a combination therapy comprises a combination of the compound of Formula (I), or a pharmaceutically acceptable salt thereof, and at least one DNA damaging agent (e.g., alkylating agents, DNA strand-breaking agents, and crosslinking agents.), or a pharmaceutically acceptable salt thereof. In some embodiments, a combination therapy consists essentially of a combination of (a) the compound of Formula (I), or a pharmaceutically acceptable salt thereof, and (b) at least one DNA damaging agent, or a pharmaceutically acceptable salt thereof (e.g., alkylating agents, DNA strand-breaking agents, and crosslinking agents.).

The term “fixed combination” means that the compound of Formula (I), or a pharmaceutically acceptable salt thereof, and an additional therapeutic agent (e.g., at least one DNA damaging agent, or a pharmaceutically acceptable salt thereof), are each administered to a subject simultaneously in the form of a single composition or dosage.

The term “non-fixed combination” means that the compound of Formula (I), or a pharmaceutically acceptable salt thereof, and an additional therapeutic agent (e.g., at least one DNA damaging agent, or a pharmaceutically acceptable salt thereof) are formulated as separate compositions or dosages such that they may be administered to a subject in need thereof concurrently or sequentially with variable intervening time limits, wherein such administration provides effective levels of the two or more compounds in the body of the subject. These also apply to cocktail therapies, e.g., the administration of three or more active ingredients.

As can be appreciated in the art, a combination therapy can be administered to a subject for a period of time. In some embodiments, the period of time occurs following the administration of a different therapeutic treatment/agent or a different combination of therapeutic treatments/agents to the subject. In some embodiments, the period of time occurs before the administration of a different therapeutic treatment/agent or a different combination of therapeutic treatments/agents to the subject.

A suitable period of time can be determined by one skilled in the art (e.g., a physician). As can be appreciated in the art, a suitable period of time can be determined by one skilled in the art based on one or more of: the stage of disease in the subject, the mass and sex of the subject, clinical trial guidelines (e.g., those on the fda.gov website), and information on the approved drug label. In some embodiments, a suitable period of time can be from 1 week to 2 years, for example, 1 week, 2, weeks, 4 weeks, 6 weeks, 8 weeks, 12 weeks, 16 weeks, 6 months, 9 months, 12 months, 18 months, or 2 years, or any value in between. In other embodiments, a suitable period of time can be from 1 month to 10 years, for example, 1 month, 6 months, 1 year, 18 months, 2 years, 3 years, 4 years, 5 years, 6 years, 7 years, 8 years, 9 years, or 10 years, or any value in between

The phrases “prior to a period of time” or “before a period of time” refer to (1) the completion of administration of treatment to the subject before the first administration of a therapeutic agent during the period of time, and/or (2) the administration of one or more therapeutic agents to the subject before a first administration of a therapeutic agent in the combination therapy described herein during the period of time, such that the one or more therapeutic agents are present in subtherapeutic and/or undetectable levels in the subject at the time the first administration of a therapeutic agent in the combination therapy is performed during the period of time. In some embodiments, the phrase “prior to a period of time” or “before a period of time” refer to the administration of one or more therapeutic agents to the subject before a first administration of a therapeutic agent in the combination therapy during the period of time, such that the one or more therapeutic agents are present in subtherapeutic levels in the subject at the time the first administration of a therapeutic agent in the combination therapy is performed during the period of time. In some embodiments, the phrase “prior to a period of time” or “before a period of time” refer to the administration of one or more therapeutic agents to the subject before a first administration of a therapeutic agent in the combination therapy during the period of time, such that the one or more therapeutic agents are present in undetectable levels in the subject at the time the first administration of a therapeutic agent in the combination therapy is performed during the period of time. In some embodiments, the phrase “prior to a period of time” or “before a period of time” refer to the administration of one or more therapeutic agents to the subject before a first administration of a therapeutic agent in the combination therapy during the period of time, such that the one or more therapeutic agents are present in subtherapeutic and/or undetectable levels in the subject at the time the first administration of a therapeutic agent in the combination therapy is performed during the period of time.

In some embodiments, a combination of (a) the compound of Formula (I), or a pharmaceutically acceptable salt thereof, and (b) at least one DNA damaging agent, or a pharmaceutically acceptable salt thereof, produces a synergistic effect; for example, any of the beneficial or desired results including clinical results as described herein, for example a decrease in tumor size, slowing of tumor growth, increased patient survival, increased tumor cell death, and reduced inhibition of tumor cell death, which is greater than the sum of effect observed when the compound of Formula (I), or a pharmaceutically acceptable salt thereof, and the at least one DNA damaging agent, or a pharmaceutically acceptable salt thereof are each administered alone.

In some embodiments, a combination of (a) the compound of Formula (I), or a pharmaceutically acceptable salt thereof, and (b) at least one DNA damaging agent, or a pharmaceutically acceptable salt thereof, produces a synergistic effect; for example, any of the beneficial or desired results including clinical results as described herein, for example decreasing tumor size, which is greater than the sum of effect observed when the same amount of the compound of Formula (I), or a pharmaceutically acceptable salt thereof, and the same amount of the at least one DNA damaging agent, or a pharmaceutically acceptable salt thereof as in the combination are each administered alone.

In some more particular embodiments a combination of (a) the compound of Formula (I), or a pharmaceutically acceptable salt thereof, and (b) at least one DNA damaging agent, or a pharmaceutically acceptable salt thereof, produces a synergistic effect, for example, a therapeutic effect using a smaller dose of either or both of (a) and (b), compared to the amount used in monotherapy. In some embodiments, the dose of (a), administered in combination with (b) may be about 0.5% to about 90% of the dose of (a) administered as a monotherapy to produce the same therapeutic effect, e.g., any of the beneficial or desired results including clinical results as described herein, for example decreasing tumor size or slowing cancer growth. In some embodiments, the dose of (a) administered in combination with (b), may be about 0.5% to 30%, about 30% to about 60%, about 60% to about 90%, such as about 0.5%, about 5%, about 10%, about 15%, about 20%, about 25%, about 30%, about 35%, about 40%, about 45%, about 50%, about 55%, about 60%, about 65%, about 70%, about 75%, about 80%, about 85%, or about 90% of the dose of (a) administered as a monotherapy. As another example, the dose of the (b) administered in combination with (a) may be about 0.5% to about 90% of the dose of (b) administered as a monotherapy to produce the same therapeutic effect, e.g., any of the beneficial or desired results including clinical results as described herein, for example decreasing tumor size or slowing cancer growth.

In some embodiments of the methods or combinations herein, a subject may be administered an amount of a compound that produces a therapeutic effect in the absence of another compound of the combinations disclosed herein. In particular embodiments of the methods or combinations herein, a subject may be administered two compounds which together produce a therapeutic effect. For example, two compounds when dosed together may have an additive or synergistic effect, such that the dose of each individual compound may independently be an effective amount, or may be a sub-therapeutic amount, but together the total amount of the combination of compounds provides a therapeutically effective amount.

In some embodiments, the amounts of the two or more compounds as provided herein together are effective in treating cancer (e.g., the amounts of the compound of Formula (I) and at least one DNA damaging agent (or a pharmaceutically acceptable salt of any of the foregoing) together are effective in treating cancer). For example, wherein the amounts of (a) the compound of Formula (I), or a pharmaceutically acceptable salt thereof, (b) at least one DNA damaging agent, or a pharmaceutically acceptable salt thereof, together are effective in treating cancer, the therapeutic effect of the combination of (a) and (b) is 10%-100% greater than, such as 10%-50%, 20%-60%, 30%-70%, 40%-80%, 50%-90%, or 60%-100%, greater than, such as 10%, 20%, 30%, 40%, 50%, 60%, 70%, 80%, or 90% greater than, the therapeutic effect of the compound of Formula (I), or a pharmaceutically acceptable salt thereof, alone. In some embodiments, wherein the amounts of (a) and (b) are effective in treating cancer, the therapeutic effect of the combination of (a) and (b) is 10%-100% greater than, such as 10%-50%, 20%-60%, 30%-70%, 40%-80%, 50%-90%, or 60%-100%, greater than, such as 10%, 20%, 30%, 40%, 50%, 60%, 70%, 80%, or 90% greater than, the therapeutic effect of (a) alone, or (b) alone (i.e., administered as a monotherapy).

In some more particular embodiments, a combination of (a) the compound of Formula (I), or a pharmaceutically acceptable salt thereof, and (b) at least one DNA damaging agent, or a pharmaceutically acceptable salt thereof, produces a synergistic effect: the desired therapeutic effect and a reduction in an unwanted drug effect, side effect, or adverse event.

In some more particular embodiments, a combination of (a) the compound of Formula (I), or a pharmaceutically acceptable salt thereof, and (b) at least one DNA damaging agent, or a pharmaceutically acceptable salt thereof, produces a synergistic effect: a therapeutic effect greater than the sum of the effect of each agent when administered alone, without any increase in an unwanted drug effect, side effect, or adverse event.

In some embodiments, the desired therapeutic effect is the same therapeutic effect observed in monotherapy of the compound of Formula (I), or a pharmaceutically acceptable salt thereof, at least one DNA damaging agent, or a pharmaceutically acceptable salt thereof, e.g., any of the beneficial or desired results including clinical results as described herein, for example decreasing tumor size or slowing cancer growth.

In some embodiments, an unwanted drug effect, side effect, or adverse event is associated with or observed in monotherapy of the compound of Formula (I), or a pharmaceutically acceptable salt thereof, or at least one DNA damaging agent, or a pharmaceutically acceptable salt thereof. For example, an unwanted drug effect, side effect, or adverse event includes, but is not limited to edema, weight loss, hypertension, cardiovascular disease, cardiovascular events (e.g., cardiovascular death, nonfatal myocardial infarction and nonfatal stroke), nausea, vomiting, diarrhea, and combinations thereof.

### Methods and Combination Therapies

The present disclosure relates to methods and combination therapies for treating cancer in a subject in need thereof by administering (a) the compound of Formula (I):

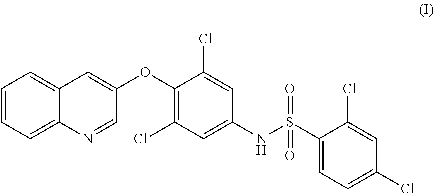 [SMILES: O=S(=O)(Nc1cc(Cl)c(Oc2cnc3ccccc3c2)c(Cl)c1)c1ccc(Cl)cc1Cl]

or a pharmaceutically acceptable salt thereof, and (b) at least one DNA damaging agent, or a pharmaceutically acceptable salt thereof. In some embodiments, the at least one DNA damaging agent, or a pharmaceutically acceptable salt thereof, is an alkylating agent. In other embodiments, the at least one DNA damaging agent, or a pharmaceutically acceptable salt thereof, is a DNA strand-breaking agent (including topoisomerase inhibitors). In still other embodiments, the at least one DNA damaging agent, or a pharmaceutically acceptable salt thereof, is a crosslinking agent.

Provided herein are methods of treating cancer in a subject in need thereof comprising or consisting essentially of administering to the subject (a) the compound of Formula (I), or a pharmaceutically acceptable salt thereof, and (b) at least one DNA damaging agent, or a pharmaceutically acceptable salt thereof, wherein the amounts of (a) and (b) together are effective in treating cancer. In some embodiments, a method of treating cancer in a subject in need thereof comprises or consists essentially of administering to the subject (a) the compound of Formula (I), or a pharmaceutically acceptable salt thereof, and (b) at least one DNA damaging agent, or a pharmaceutically acceptable salt thereof, during a period of time, wherein the amounts of (a) and (b) together are effective in treating cancer.

Also provided herein are methods of treating a subject, the method comprising: selecting a subject having cancer; and administering (a) the compound of Formula (I), or a pharmaceutically acceptable salt thereof, and (b) at least one DNA damaging agent, or a pharmaceutically acceptable salt thereof, to the selected subject, wherein the amounts of (a) and (b) together are effective in treating cancer. In some embodiments, (a) and (b) are administered during a period of time.

Also provided herein are methods of treating a subject, the method comprising: identifying a subject having cancer; and administering (a) the compound of Formula (I), or a pharmaceutically acceptable salt thereof, and (b) at least one DNA damaging agent, or a pharmaceutically acceptable salt thereof, to the selected subject, wherein the amounts of (a) and (b) together are effective in treating cancer. In some embodiments, (a) and (b) are administered during a period of time.

In some embodiments, the subject that has been identified, selected, or diagnosed as having cancer through the use of histological analysis and/or a regulatory agency-approved, e.g., FDA-approved test or assay for identifying cancer in a subject or a biopsy sample from the subject or by performing any of the non-limiting examples of assays described herein. In some embodiments, the test or assay is provided as a kit. The sample can be a tissue biopsy or a liquid biopsy. In some embodiments, the assay is selected from next generation sequencing, immunohistochemistry, fluorescence microscopy, Southern blotting, Western blotting, Northern blotting, FACS analysis, and PCR-based amplification.

Also provided herein are methods of inhibiting cancer cell proliferation in a subject in need thereof, the method comprising contacting the cancer cell with (a) a therapeutically effective amount of the compound of Formula (I), or a pharmaceutically acceptable salt thereof, and (b) a therapeutically effective amount of at least one DNA damaging agent, or a pharmaceutically acceptable salt thereof.

Also provided herein are methods of treating cancer in a subject in need thereof consisting essentially of (a) administering to the subject the compound of Formula (I), or a pharmaceutically acceptable salt thereof, and (b) one DNA damaging agent, or a pharmaceutically acceptable salt thereof, selected from: cyclophosphamide, mechlorethamine, chlorambucil, melphalan, dacarbazine, temozolomide, cisplatin, carboplatin, oxaliplatin, nedaplatin, triplatin tetranitrate, phenanthriplatin, picoplatin, satraplatin, carmustine, lomustine, semustine, bendamustine, nimustine, ranimustine, uramustine, busulfan, chlormethine, fotemustine, ifosfamide, streptozocin, bleomycin, mitomycin, altretamine, irinotecan, topotecan, camptothecin, lamellarin, etoposide, teniposide, doxorubicin, daunorubicin, epirubicin, idarubicin, valrubicin, tafluposide mitoxantrone, and amsacrine; and pharmaceutically acceptable salts thereof; wherein the amounts of (a) and (b) together are effective in treating cancer.

In some embodiments, the cancer can be identified using a liquid biopsy (variously referred to as a fluid biopsy or fluid phase biopsy). See, e.g., Karachialiou et al., Ann. Transl. Med., 3(3):36, 2016. Liquid biopsy methods can be used to detect, for example, total tumor burden and/or the presence of a particular cancer type. Liquid biopsies can be performed on biological samples obtained relatively easily from a subject (e.g., via a simple blood draw) and are generally less invasive than traditional methods. In some embodiments, liquid biopsies can be used to detect the presence of cancer at an earlier stage than traditional methods. In some embodiments, the biological sample to be used in a liquid biopsy can include, CSF, blood, plasma, urine, saliva, sputum, broncho-alveolar lavage, bile, lymphatic fluid, cyst fluid, stool, ascites, and combinations thereof. In some embodiments, a liquid biopsy can be used to detect circulating tumor cells (CTCs). In some embodiments, a liquid biopsy can be used to detect cell-free DNA. In some embodiments, cell-free DNA detected using a liquid biopsy is circulating tumor DNA (ctDNA) that is derived from tumor cells. Analysis of ctDNA (e.g., using sensitive detection techniques such as, without limitation, next-generation sequencing (NGS), traditional PCR, digital PCR, or microarray analysis) can be used to identify particular cancer types.

Liquid biopsies can be performed at multiple times during a course of diagnosis, a course of monitoring, and/or a course of treatment to determine one or more clinically relevant parameters including, without limitation, progression of the disease or efficacy of a treatment, after administering a treatment to the subject. For example, a first liquid biopsy can be performed at a first time point and a second liquid biopsy can be performed at a second time point during a course of diagnosis, a course of monitoring, and/or a course of treatment. In some embodiments, the first time point can be a time point prior to diagnosing a subject with a disease (e.g., when the subject is healthy), and the second time point can be a time point after subject has developed the disease (e.g., the second time point can be used to diagnose the subject with the disease). In some embodiments, the first time point can be a time point prior to diagnosing a subject with a disease (e.g., when the subject is healthy), after which the subject is monitored, and the second time point can be a time point after monitoring the subject. In some embodiments, the first time point can be a time point after diagnosing a subject with a disease, after which a treatment is administered to the subject, and the second time point can be a time point after the treatment is administered; in such cases, the second time point can be used to assess the efficacy of the treatment (e.g., if the genetic mutation(s) detected at the first time point are reduced in abundance or are undetectable). In some embodiments, a treatment to be administered to a subject can include a compound of Formula I, or a pharmaceutically acceptable salt thereof, and at least one DNA damaging agent, or a pharmaceutically acceptable salt thereof.

In some embodiments, the cancer is selected from colorectal cancer, breast cancer, esophageal cancer, gastric cancer, pancreatic cancer, liver cancer, adrenocortical cancer, prostate cancer, liposarcoma, thyroid cancer, bladder cancer, kidney cancer, melanoma, squamous cell carcinoma, cervical cancer, testicular cancer, neuroblastoma, small cell lung cancer, non-small cell lung cancer, glioblastoma multiforme, astrocytoma, ovarian cancer, glioma, medulloblastoma, Hodgkin's lymphoma, non-Hodgkin's lymphoma, lymphocytic lymphoma (nodular or diffuse), mixed-cell type lymphoma, histocytic lymphoma, Burkitt's lymphoma, AIDS-related Kaposi's sarcoma, multiple myeloma, and a leukemia such as, for example, chronic lymphocytic leukemia, chronic granulocytic leukemia, acute myelogenous leukemia, and acute lymphoblastic leukemia.

In some embodiments, the cancer is a leukemia, for example chronic lymphocytic leukemia, chronic granulocytic leukemia, acute myelogenous leukemia, or acute lymphoblastic leukemia. In other embodiments, the cancer is cervical cancer, testicular cancer, or ovarian cancer. In still other embodiments, the cancer is small cell lung cancer, non-small cell lung cancer, colorectal cancer, or breast cancer. In yet still other embodiments, the cancers is a brain cancer, for example, neuroblastoma, glioblastoma multiforme, astrocytoma, glioma, or medulloblastoma. In some embodiments, the cancer is a lymphoma, for example, Hodgkin's lymphoma, non-Hodgkin's lymphoma, lymphocytic lymphoma (nodular or diffuse), mixed-cell type lymphoma, histocytic lymphoma, or Burkitt's lymphoma. In other embodiments, the cancer is a skin cancer, for example, melanoma or squamous cell carcinoma. In still other embodiments, the cancer is esophageal cancer, gastric cancer, pancreatic cancer, liver cancer, adrenocortical cancer, prostate cancer, liposarcoma, thyroid cancer, bladder cancer, kidney cancer, AIDS-related Kaposi's sarcoma, or multiple myeloma.

In some embodiments, the cancer is colorectal cancer. In other embodiments, the cancer is breast cancer. In still other embodiments, the cancer is esophageal cancer. In yet still other embodiments, the cancer is gastric cancer. In some embodiments, the cancer is pancreatic cancer. In other embodiments, the cancer is liver cancer. In still other embodiments, the cancer is adrenocortical cancer. In yet still other embodiments, the cancer is prostate cancer. In some embodiments, the cancer is liposarcoma. In other embodiments, the cancer is thyroid cancer. In still other embodiments, the cancer is bladder cancer. In yet still other embodiments, the cancer is kidney cancer. In some embodiments, the cancer is melanoma. In other embodiments, the cancer is squamous cell carcinoma. In still other embodiments, the cancer is cervical cancer. In yet still other embodiments, the cancer is testicular cancer. In some embodiments, the cancer is neuroblastoma. In some embodiments, the cancer is small cell lung cancer. In other embodiments, the cancer is non-small cell lung cancer. In still other embodiments, the cancer is glioblastoma multiforme. In yet still other embodiments, the cancer is astrocytoma. In some embodiments, the cancer is ovarian cancer. In other embodiments, the cancer is glioma. In still other embodiments, the cancer is medulloblastomas. In yet still other embodiments, the cancer is Hodgkin's lymphoma. In some embodiments, the cancer is non-Hodgkin's lymphoma. In other embodiments, the cancer is lymphocytic lymphoma (nodular or diffuse). In still other embodiments, the cancer is mixed-cell type lymphoma. In yet still other embodiments, the cancer is histocytic lymphoma. In some embodiments, the cancer is Burkitt's lymphoma. In other embodiments, the cancer is multiple myeloma. In still other embodiments, the cancer is chronic lymphocytic leukemia. In yet still other embodiments, the cancer is chronic granulocytic leukemia. In some embodiments, the cancer is acute myelogenous leukemia. In other embodiments, the cancer is acute lymphoblastic leukemia. In still other embodiments, the cancer is AIDS-related Kaposi's sarcoma.

In some embodiments, (a) and (b) are administered concurrently. In some embodiments, (a) and (b) are administered as a fixed combination. In some embodiments, (a) and (b) are administered as a non-fixed combination. In some embodiments, (a) and (b) are administered sequentially and in any order, at specific or varying time intervals (e.g., during the period of time). In some embodiments, a therapeutically effective amount of each of (a) and (b) are administered concurrently. In some embodiments, a therapeutically effective amount of each of (a) and (b) are administered sequentially and in any order, at specific or varying time intervals (e.g., during the period of time).

The compound of Formula (I), or a pharmaceutically acceptable salt thereof, and the at least one DNA damaging agent, or a pharmaceutically acceptable salt thereof, may be administered in one or more doses, or as part of the same or separate dosage forms, via the same or different routes of administration, and/or on the same or different administration schedules according to standard pharmaceutical practice known to one skilled in the art.

In some embodiments, the amount of the compound of Formula (I), or a pharmaceutically acceptable salt thereof, is from about 0.1 to about 25 milligrams (mg), or any value in between. For example, from about 0.1 mg to about 10 mg, about 5 mg to about 15 mg, about 10 mg to about 20 mg, or about 15 mg to about 25 mg. In some embodiments, the compound of Formula (I), or a pharmaceutically acceptable salt thereof, is administered at a dose of about 1 mg to about 15 mg. In some embodiments, the compound of Formula (I), or a pharmaceutically acceptable salt thereof, is administered at a dose of about 0.5 mg, 0.75 mg, 1.0 mg, 1.25 mg, 1.5 mg, 1.75 mg, 2.0 mg, 2.25 mg, 2.5 mg, 2.75 mg, 3.0 mg, 3.25 mg, 3.5 mg, 3.75 mg, 4.0 mg, 4.25 mg, 4.5 mg, 4.75 mg, 5.0 mg, 5.5 mg, 6.0 mg, 6.5 mg, 7.0 mg, 7.5 mg, 8.0 mg, 8.5 mg, 9.0 mg, 9.5 mg, 10.0 mg, 10.5 mg, 11.0 mg, 11.5 mg, 12.0 mg, 12.5 mg, 13.0 mg, 13.5 mg, 14.0 mg, 14.5 mg, or 15.0 mg. In some embodiments, the compound of Formula (I), or a pharmaceutically acceptable salt thereof, is administered at a dose of about 1 mg to about 5 mg, about 5 mg to about 10 mg, or about 10 mg to about 15 mg. In some embodiments, the compound of Formula (I), or a pharmaceutically acceptable salt thereof, is administered at a dose of about 2 mg to about 6 mg. In some embodiments, the compound of Formula (I), or a pharmaceutically acceptable salt thereof, is administered at a dose of about 1 mg, about 2 mg, or about 3 mg. In some embodiments, the dose is a therapeutically effective amount.

In some embodiments, the compound of Formula (I), or a pharmaceutically acceptable salt thereof, is administered to the subject three times a day, twice a day, daily, every other day, three times a week, twice a week, weekly, every other week, twice a month, or monthly. In some embodiments, the compound of Formula (I), or a pharmaceutically acceptable salt thereof, is administered to the subject daily.

In some embodiments, the compound of Formula (I), or a pharmaceutically acceptable salt thereof, is administered to the subject before administration of the at least one DNA damaging agent, or a pharmaceutically acceptable salt thereof, to the subject. In some embodiments, the compound of Formula (I) is administered to the subject at least 14, 13, 12, 11, 10, 9, 8, 7, 6, 5, 4, 3, 2, or 1 day(s) before administration of the at least one DNA damaging agent, or a pharmaceutically acceptable salt thereof. In some embodiments, the compound of Formula (I) is administered to the subject 14, 13, 12, 11, 10, 9, 8, 7, 6, 5, 4, 3, 2, or 1 day(s) before administration of the at least one DNA damaging agent, or a pharmaceutically acceptable salt thereof. In a further embodiment, the compound of Formula (I), or a pharmaceutically acceptable salt thereof, is administered to the subject daily.

In some embodiments, the compound of Formula (I), or a pharmaceutically acceptable salt thereof, is administered to the subject before and concurrently with administration of the at least one DNA damaging agent, or a pharmaceutically acceptable salt thereof, to the subject. In some more particular embodiments, the compound of Formula (I) is administered to the subject at least 14, 13, 12, 11, 10, 9, 8, 7, 6, 5, 4, 3, 2, or 1 day(s) before, and concurrently with, administration of the at least one DNA damaging agent, or a pharmaceutically acceptable salt thereof. In other more particular embodiments, the compound of Formula (I) is administered to the subject 14, 13, 12, 11, 10, 9, 8, 7, 6, 5, 4, 3, 2, or 1 day(s) before, and concurrently with, administration of the at least one DNA damaging agent, or a pharmaceutically acceptable salt thereof. In a further embodiment, the compound of Formula (I), or a pharmaceutically acceptable salt thereof, is administered to the subject daily.

In some embodiments, the compound of Formula (I), or a pharmaceutically acceptable salt thereof, is administered to the subject before, concurrently with, and after administration of the at least one DNA damaging agent, or a pharmaceutically acceptable salt thereof, to the subject. In some more particular embodiments, the compound of Formula (I) is administered to the subject at least 14, 13, 12, 11, 10, 9, 8, 7, 6, 5, 4, 3, 2, or 1 day(s) before, concurrently with, and after, administration of the at least one DNA damaging agent, or a pharmaceutically acceptable salt thereof. In other more particular embodiments, the compound of Formula (I) is administered to the subject 14, 13, 12, 11, 10, 9, 8, 7, 6, 5, 4, 3, 2, or 1 day(s) before, concurrently with, and after, administration of the at least one DNA damaging agent, or a pharmaceutically acceptable salt thereof. In some embodiments, the compound of Formula (I) is administered to the subject for at least 14, 13, 12, 11, 10, 9, 8, 7, 6, 5, 4, 3, 2, or 1 day(s) after administration of the at least one DNA damaging agent, or a pharmaceutically acceptable salt thereof. In still other more particular embodiments, the compound of Formula (I) is administered to the subject for 14, 13, 12, 11, 10, 9, 8, 7, 6, 5, 4, 3, 2, or 1 day(s) after administration of the at least one DNA damaging agent, or a pharmaceutically acceptable salt thereof. In a further embodiment, the compound of Formula (I), or a pharmaceutically acceptable salt thereof, is administered to the subject daily.

In some embodiments, the compound of Formula (I) is in the form of a besylate salt. In some embodiments, the compound of Formula (I) is in the form of an HCl salt. In some embodiments, the compound of Formula (I) is in the form of an HBr salt. In some embodiments, the compound of Formula (I) is in the form of a tosylate salt.

In some embodiments, the at least one DNA damaging agent, or a pharmaceutically acceptable salt thereof, is a platinum-based agent (such as cisplatin or carboplatin). In other embodiments, the at least one DNA damaging agent, or a pharmaceutically acceptable salt thereof, is an anthracycline. In still other embodiments, the at least one DNA damaging agent, or a pharmaceutically acceptable salt thereof, is a topoisomerase II inhibitor (such as doxorubicin or daunorubicin).

In some embodiments, the at least one DNA damaging agent, or a pharmaceutically acceptable salt thereof, is selected from the group consisting of: cyclophosphamide, mechlorethamine, chlorambucil, melphalan, dacarbazine, temozolomide, cisplatin, carboplatin, oxaliplatin, nedaplatin, triplatin tetranitrate, phenanthriplatin, picoplatin, satraplatin, carmustine, lomustine, semustine, bendamustine, nimustine, ranimustine, uramustine, busulfan, chlormethine, fotemustine, ifosfamide, streptozocin, bleomycin, mitomycin, altretamine, irinotecan, topotecan, camptothecin, lamellarin, etoposide, teniposide, doxorubicin, daunorubicin, epirubicin, idarubicin, valrubicin, tafluposide mitoxantrone, and amsacrine; and pharmaceutically acceptable salts thereof.

In some embodiments, the at least one DNA damaging agent, or a pharmaceutically acceptable salt thereof, is a single DNA damaging agent selected from the group consisting of: cyclophosphamide, mechlorethamine, chlorambucil, melphalan, dacarbazine, temozolomide, cisplatin, carboplatin, oxaliplatin, nedaplatin, triplatin tetranitrate, phenanthriplatin, picoplatin, satraplatin, carmustine, lomustine, semustine, bendamustine, nimustine, ranimustine, uramustine, busulfan, chlormethine, fotemustine, ifosfamide, streptozocin, bleomycin, mitomycin, altretamine, irinotecan, topotecan, camptothecin, lamellarin, etoposide, teniposide, doxorubicin, daunorubicin, epirubicin, idarubicin, valrubicin, tafluposide mitoxantrone, and amsacrine; and pharmaceutically acceptable salts thereof.

In some embodiments, the at least one DNA damaging agent is cyclophosphamide, or a pharmaceutically acceptable salt thereof. In other embodiments, the at least one DNA damaging agent is mechlorethamine, or a pharmaceutically acceptable salt thereof. In still other embodiments, the at least one DNA damaging agent is chlorambucil, or a pharmaceutically acceptable salt thereof. In yet still other embodiments, the at least one DNA damaging agent is melphalan, or a pharmaceutically acceptable salt thereof. In some embodiments, the at least one DNA damaging agent is dacarbazine, or a pharmaceutically acceptable salt thereof. In other embodiments, the at least one DNA damaging agent is temozolomide, or a pharmaceutically acceptable salt thereof. In other embodiments, the at least one DNA damaging agent is cisplatin, or a pharmaceutically acceptable salt thereof. In still other embodiments, the at least one DNA damaging agent is carboplatin, or a pharmaceutically acceptable salt thereof. In yet still other embodiments, the at least one DNA damaging agent is oxaliplatin, or a pharmaceutically acceptable salt thereof. In some embodiments, the at least one DNA damaging agent is nedaplatin, or a pharmaceutically acceptable salt thereof. In other embodiments, the at least one DNA damaging agent is triplatin tetranitrate, or a pharmaceutically acceptable salt thereof. In other embodiments, the at least one DNA damaging agent is phenanthriplatin, or a pharmaceutically acceptable salt thereof. In still other embodiments, the at least one DNA damaging agent is picoplatin, or a pharmaceutically acceptable salt thereof. In yet still other embodiments, the at least one DNA damaging agent is satraplatin, or a pharmaceutically acceptable salt thereof. In some embodiments, the at least one DNA damaging agent is carmustine, or a pharmaceutically acceptable salt thereof. In other embodiments, the at least one DNA damaging agent is lomustine, or a pharmaceutically acceptable salt thereof. In still other embodiments, the at least one DNA damaging agent is semustine, or a pharmaceutically acceptable salt thereof. In yet still other embodiments, the at least one DNA damaging agent is bendamustine, or a pharmaceutically acceptable salt thereof. In some embodiments, the at least one DNA damaging agent is imustine, or a pharmaceutically acceptable salt thereof. In other embodiments, the at least one DNA damaging agent is ranimustine, or a pharmaceutically acceptable salt thereof. In still other embodiments, the at least one DNA damaging agent is uramustine, or a pharmaceutically acceptable salt thereof. In yet still other embodiments, the at least one DNA damaging agent is busulfan, or a pharmaceutically acceptable salt thereof. In some embodiments, the at least one DNA damaging agent is chlormethine, or a pharmaceutically acceptable salt thereof. In other embodiments, the at least one DNA damaging agent is fotemustine, or a pharmaceutically acceptable salt thereof. In yet still other embodiments, the at least one DNA damaging agent is ifosfamide, or a pharmaceutically acceptable salt thereof. In some embodiments, the at least one DNA damaging agent is streptozocin, or a pharmaceutically acceptable salt thereof. In other embodiments, the at least one DNA damaging agent is bleomycin, or a pharmaceutically acceptable salt thereof. In some embodiments, the at least one DNA damaging agent is itomycin, or a pharmaceutically acceptable salt thereof. In other embodiments, the at least one DNA damaging agent is altretamine, or a pharmaceutically acceptable salt thereof. In some embodiments, the at least one DNA damaging agent is irinotecan, or a pharmaceutically acceptable salt thereof. In other embodiments, the at least one DNA damaging agent is topotecan, or a pharmaceutically acceptable salt thereof. In still other embodiments, the at least one DNA damaging agent is camptothecin, or a pharmaceutically acceptable salt thereof. In some embodiments, the at least one DNA damaging agent is lamellarin, or a pharmaceutically acceptable salt thereof. In other embodiments, the at least one DNA damaging agent is etoposide, or a pharmaceutically acceptable salt thereof. In still other embodiments, the at least one DNA damaging agent is teniposide, or a pharmaceutically acceptable salt thereof. In some embodiments, the at least one DNA damaging agent is doxorubicin, or a pharmaceutically acceptable salt thereof. In other embodiments, the at least one DNA damaging agent is daunorubicin, or a pharmaceutically acceptable salt thereof. In still other embodiments, the at least one DNA damaging agent is epirubicin, or a pharmaceutically acceptable salt thereof. In some embodiments, the at least one DNA damaging agent is idarubicin, or a pharmaceutically acceptable salt thereof. In other embodiments, the at least one DNA damaging agent is valrubicin, or a pharmaceutically acceptable salt thereof. In still other embodiments, the at least one DNA damaging agent is tafluposide, or a pharmaceutically acceptable salt thereof. In some embodiments, the at least one DNA damaging agent is mitoxantrone, or a pharmaceutically acceptable salt thereof. In other embodiments, the at least one DNA damaging agent is amsacrine, or a pharmaceutically acceptable salt thereof.

In some embodiments, the at least one DNA damaging agent, or a pharmaceutically acceptable salt thereof, is two DNA damaging agents selected from the group consisting of: cyclophosphamide, mechlorethamine, chlorambucil, melphalan, dacarbazine, temozolomide, cisplatin, carboplatin, oxaliplatin, nedaplatin, triplatin tetranitrate, phenanthriplatin, picoplatin, satraplatin, carmustine, lomustine, semustine, bendamustine, nimustine, ranimustine, uramustine, busulfan, chlormethine, fotemustine, ifosfamide, streptozocin, bleomycin, mitomycin, altretamine, irinotecan, topotecan, camptothecin, lamellarin, etoposide, teniposi de, doxorubicin, daunorubicin, epirubicin, idarubicin, valrubicin, tafluposide mitoxantrone, and amsacrine; and pharmaceutically acceptable salts thereof.

In some embodiments, the amount of the compound of Formula (I), or a pharmaceutically acceptable salt thereof, is from about 1 mg to about 350 mg, or any value in between. For example, about 1 mg to about 175 mg, about 175 mg to about 350 mg, about 90 mg to about 260 mg, or about 150 mg to about 200 mg.

In some embodiments, the amount of the at least one DNA damaging agent, or a pharmaceutically acceptable salt thereof, is from about 1 mg to about 500 mg, or any value in between. For example, about 1 mg to about 250 mg, about 250 mg to about 500 mg, about 50 mg to about 250 mg, or about 100 mg to about 200 mg. The particular dose of the at least one DNA damaging agent, or a pharmaceutically acceptable salt thereof, that is required for a subject can readily be determined by one skilled in the art, for example, by applying body surface area-dosing, calculations based on glomerular filtration rate, or other methods known in the art.

In some embodiments, the at least one DNA damaging agent, or a pharmaceutically acceptable salt thereof, is administered to the subject three times a day, twice a day, daily, every other day, three times a week, twice a week, weekly, every other week, twice a month, or monthly. In some embodiments, the at least one DNA damaging agent, or a pharmaceutically acceptable salt thereof, is administered to the subj ect daily. In some embodiments, the at least one DNA damaging agent, or a pharmaceutically acceptable salt thereof, is administered to the subject for a first period of time, and a second period of time, with from about one week to about four weeks in between the first period of time and the second period of time. For example, in some embodiments, the at least one DNA damaging agent, or a pharmaceutically acceptable salt thereof, is administered to the subject daily for four weeks (the first period of time), not administered for one week, and then administered for four more weeks (the second period of time).

In some embodiments, the subject has been previously treated with an anticancer therapy selected from surgery and radiotherapy. In some embodiments, the previous anticancer therapy was between about 1 hour to about 2 year ago, or any value in between. For example, a subject can undergo anticancer therapy (e.g., surgery or radiotherapy), followed by treatment with a compound of Formula I, or a pharmaceutically acceptable salt thereof, and at least one DNA damaging agent, 1 hour later, 2 hours later, 4 hours later, 6 hours later, 12 hours later, 24 hours later, 2 days later, one week later, two weeks later, one month later, two months later, three months later, six months later, 12 months later, 18 months later, or 24 months later, or any value in between. In other embodiments, the subject is treated with an anticancer therapy selected from surgery and radiotherapy after at least one administration of a compound of Formula I, or a pharmaceutically acceptable salt thereof, and at least one DNA damaging agent or a pharmaceutically acceptable salt thereof. In some embodiments, the compound of Formula I, or a pharmaceutically acceptable salt thereof, and at least one DNA damaging agent or a pharmaceutically acceptable salt thereof is administered to the subject between about 1 hour to about 2 year after the anticancer therapy, or any value in between. For example, after administration of a compound of Formula I, or a pharmaceutically acceptable salt thereof, and at least one DNA damaging agent or a pharmaceutically acceptable salt thereof, a subject can undergo anticancer therapy 1 hour later, 2 hours later, 4 hours later, 6 hours later, 12 hours later, 24 hours later, 2 days later, one week later, two weeks later, one month later, two months later, three months later, six months later, 12 months later, 18 months later, or 24 months later, or any value in between.

In some embodiments, the anticancer therapy is radiotherapy. Non-limiting examples of radiotherapy include external radiation beam therapy (e.g., external beam therapy using kilovoltage X-rays or megavoltage X-rays) or internal radiotherapy. Internal radiotherapy (also called brachytherapy) can include the use of, e.g., low-dose internal radiotherapy or high-dose internal radiotherapy. Low-dose internal radiotherapy includes, e.g., inserting small radioactive pellets (also called seeds) into or proximal to a cancer tissue in the subject. High-dose internal radiotherapy includes, e.g., inserting a thin tube (e.g., a catheter) or an implant into or proximal to a cancer tissue in the subject, and delivering a high dose of radiation to the thin tube or implant using a radiation machine. Methods for performing radiotherapy on a subject having a cancer are known in the art.

In other embodiments, the anticancer therapy is surgery. Non-limiting examples of surgery include, e.g., open surgery or minimally invasive surgery. Surgery can include, e.g., at least a partial resection of the tumor, removing an entire tumor, debulking of a tumor, or removing a tumor that is causing pain or pressure in the subject. Methods for performing open surgery and minimally invasive surgery on a subject having a cancer are known in the art.

### EXAMPLES

The following examples further illustrate the invention. For example, the efficacy of CHS-131, in combination with at least one DNA damaging agent, or a pharmaceutically acceptable salt thereof, to treat cancer is demonstrated in the following examples. Procedures are similar to those described in Khandekar, et al., Proc. Nat. Acad. Sci. USA, Vol. 115, No. 3, pp. 561-566 (2018), which is hereby incorporated by reference in its entirety, including the supplemental information.

### Example 1

The ability of CHS-131 to increase tumor cell death, in combination with chemotherapeutic agents, by inhibiting PPARγ phosphorylation is evaluated in tumor cell lines.

Antibodies are obtained from commercial sources unless otherwise indicated. The PPARγ IP experiments are performed using E-8 antibody from Santa Cruz. The phospho-specific antibody to pS273 PPARγ has previously been described. See Choi, et al., Nature, 466, pp. 451-456 (2010). Immortalized fibroblasts are generated by obtaining the interscapular brown adipose stromal vascular fraction from 4-wk-old mice with the following genotypes: Ppargwt/wt or PpargS273A/S273A. The tissue is dissected, washed in PBS, minced, and digested for 45 min at 37° C. The tissue suspension is filtered through a 40-μm cell strainer and centrifuged for 5 min to pellet the fibroblastic cells. The cell pellet is resuspended and plated. After 3 d of culture, a fibroblastic culture is obtained. The cells are then infected with lentivirus containing SV40 (abm). Cells are verified for infection by RT-PCR for SV40 viral antigens. These cells are maintained in DMEM+10% FBS. For lentiviral experiments, 293T cells are transfected with Fugene 6 (Roche) with viral vectors and supernatants harvested after 48 h. Cells are infected for 24 h, and analyzed 24 h after infection is complete.

RNA is extracted from cultured cells or frozen tissue samples and reverse-transcribed. Resulting cDNA is analyzed by qRT-PCR in 384-well format and relative mRNA levels are calculated using the comparative CT method normalized to TATA binding protein mRNA.

For IP, crude nuclear extracts are prepared from confluent cells grown on 15-cm plates using standard procedures. 1 g of nuclear extract protein is diluted in buffer containing 50 mM Tris, 1% Igepal CA-360, 10% glycerol, and the final concentration of NaCl is adjusted to 150 mM. PPARγ is immunoprecipitated with antibody overnight, and antigen/antibody complexes are collected using Dynabeads Protein G (ThermoFisher) and a magnetic rack. Beads are washed five times with the IP buffer, and elution is performed using NuPage LDS buffer with 2.5% β-mercaptoethanol and boiling for 5 min. Samples are run on SDS gels as described previously by Choi, et al. For Western blotting, samples are collected and lysed in RIPA buffer and run on SDS gels using standard procedures.

### Example 2

The effects of treatment with CHS-131 (compound of Formula (I)), alone and in combination with at least one DNA damaging agent, to treat cancer are evaluated in mice. Various models can be used. Subjects are divided into groups for treatment and evaluation. Groups can include, controls (e.g. subjects with or without a particular cancer that are not administered a therapy), subjects administered monotherapy (e.g. CHS-131 at two different doses, or at least one DNA damaging agent), subjects administered a combo-therapy (e.g. CHS-131 at two different doses, and at least one DNA damaging agent), and subjects administered a positive control therapy (e.g., standard of control therapy for a particular cancer). Various parameters such as tumor size, cancer progression, and development of metastases are evaluated. Each animal is administered the respective compositions (e.g. vehicle, monotherapy, combo-therapy) starting on Day 0 and ending on Day 42. Tissue and/or fluid sample (e.g., by solid or liquid biopsy or organ removal) are collected for analysis.

Six- to 10-wk-old nude male mice are randomly assigned to treatment groups of: vehicle, vehicle+carboplatin (50 mg/kg, Monday, Wednesday, Friday), CHS-131 (0.04 mg/kg, once a day)+carboplatin, CHS-131 alone (0.04 mg/kg once a day), CHS-131 (50 mg/kg, once a day)+carboplatin, or CHS-131 alone (50 mg/kg once a day). Xenografts are generated by injecting 5×10⁶ cells in DMEM media into the flank. Tumor dimensions are measured twice weekly using tumor calipers and converted to volume using the formula V=(π×length)×(width²/6). Treatment is started once tumors measure 50-75 mm3 are present on the flank. Mice are weighed daily for dosing. Treatment drugs are dissolved in vehicle containing DMSO, Cremophor EL, and sterile saline. Drugs are delivered via intraperitoneal injection twice a day. Carboplatin or sterile saline is delivered via intraperitoneal injection on Monday, Wednesday, and Friday.

Tumors or lungs are removed from animals and fixed with 4% paraformaldehyde followed by dehydration in 70% ethanol before embedding. Tissues are embedded in paraffin and immunohistochemistry was performed as described in Lo, et al., Cell, 158, pp. 41-53 (2014). TUNEL staining is performed using the Apoptag Peroxidase in Situ Apoptosis Detection Kit (EMD Millipore) per the manufacturer's instructions. Images are acquired containing the entire tissue section, and analyzed using the Aperio Imagescope Software.

1-39. (canceled)

40. A method of treating cancer in a subject in need thereof comprising administering to the subject
(a) a therapeutically effective amount of compound of Formula (I), 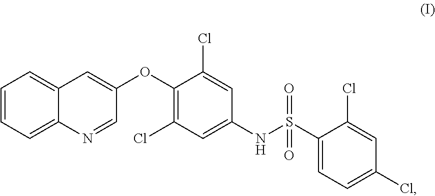 [SMILES: O=S(=O)(Nc1cc(Cl)c(Oc2cnc3ccccc3c2)c(Cl)c1)c1ccc(Cl)cc1Cl]
or a pharmaceutically acceptable salt thereof, and
(b) a therapeutically effective amount of at least one DNA damaging agent, or a pharmaceutically acceptable salt thereof.

41. The method of claim 40, wherein (a) and (b) are administered concurrently.

42. The method of claim 40, wherein (a) and (b) are administered sequentially in either order.

43. The method of claim 40, wherein the compound of Formula (I), or a pharmaceutically acceptable salt thereof, is administered at a dose from about 1 mg to about 15 mg.

44. The method of claim 40, wherein the compound of Formula (I), or a pharmaceutically acceptable salt thereof, is administered to the subject three times a day, twice a day, daily, every other day, three times a week, twice a week, weekly, every other week, twice a month, or monthly.

45. The method of claim 44, wherein the compound of Formula (I), or a pharmaceutically acceptable salt thereof, is administered to the subject daily.

46. The method of claim 40, wherein the at least one DNA damaging agent, or a pharmaceutically acceptable salt thereof, is a single DNA damaging agent selected from cyclophosphamide, mechlorethamine, chlorambucil, melphalan, dacarbazine, temozolomide, cisplatin, carboplatin, oxaliplatin, nedaplatin, triplatin tetranitrate, phenanthriplatin, picoplatin, satraplatin, carmustine, lomustine, semustine, bendamustine, nimustine, ranimustine, uramustine, busulfan, chlormethine, fotemustine, ifosfamide, streptozocin, bleomycin, mitomycin, altretamine, irinotecan, topotecan, camptothecin, lamellarin, etoposide, teniposide, doxorubicin, daunorubicin, epirubicin, idarubicin, valrubicin, tafluposide mitoxantrone, and amsacrine; and pharmaceutically acceptable salts thereof.

47. The method of claim 40, wherein the at least one DNA damaging agent, or a pharmaceutically acceptable salt thereof, is two DNA damaging agents selected from cyclophosphamide, mechlorethamine, chlorambucil, melphalan, dacarbazine, temozolomide, cisplatin, carboplatin, oxaliplatin, nedaplatin, triplatin tetranitrate, phenanthriplatin, picoplatin, satraplatin, carmustine, lomustine, semustine, bendamustine, nimustine, ranimustine, uramustine, busulfan, chlormethine, fotemustine, ifosfamide, streptozocin, bleomycin, mitomycin, altretamine, irinotecan, topotecan, camptothecin, lamellarin, etoposide, teniposide, doxorubicin, daunorubicin, epirubicin, idarubicin, valrubicin, tafluposide mitoxantrone, and amsacrine; and pharmaceutically acceptable salts thereof.

48. The method of claim 46, wherein the at least one DNA damaging agent, or a pharmaceutically acceptable salt thereof, is a single DNA damaging agent selected from cisplatin, carboplatin, and oxaliplatin.

49. The method of claim 48, wherein the at least one DNA damaging agent is cisplatin.

50. The method of claim 48, wherein the at least one DNA damaging agent is carboplatin.

51. The method of claim 48, wherein the at least one DNA damaging agent is oxaliplatin.

52. The method of claim 40, wherein the compound of Formula (I) is present as a pharmaceutically acceptable salt.

53. The method of claim 40, wherein the compound of Formula (I) is present as the besylate salt.

54. The method of claim 40, wherein the compound of Formula (I) is present as the free base.

55. The method of claim 40, wherein the cancer is selected from: colorectal cancer, breast cancer, esophageal cancer, gastric cancer, pancreatic cancer, liver cancer, adrenocortical cancer, prostate cancer, liposarcoma, thyroid cancer, bladder cancer, kidney cancer, melanoma, squamous cell carcinoma, cervical cancer, testicular cancer, neuroblastoma, small cell lung cancer, non-small cell lung cancer, glioblastoma multiforme, astrocytoma, ovarian cancer, glioma, medulloblastoma, Hodgkin's lymphoma, non-Hodgkin's lymphoma, lymphocytic lymphoma (nodular or diffuse), mixed-cell type lymphoma, histocytic lymphoma, Burkitt's lymphoma, AIDS-related Kaposi's sarcoma, multiple myeloma, chronic lymphocytic leukemia, chronic granulocytic leukemia, acute myelogenous leukemia, and acute lymphoblastic leukemia.

56. A pharmaceutical composition comprising
(a) the compound of Formula (I), 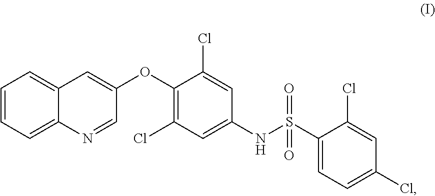 [SMILES: O=S(=O)(Nc1cc(Cl)c(Oc2cnc3ccccc3c2)c(Cl)c1)c1ccc(Cl)cc1Cl]
or a pharmaceutically acceptable salt thereof,
(b) at least one DNA damaging agent, or a pharmaceutically acceptable salt thereof, and one or more pharmaceutical excipients.

57. The pharmaceutical composition of claim 56, wherein the at least one DNA damaging agent, or a pharmaceutically acceptable salt thereof, is a single DNA damaging agent selected from cyclophosphamide, mechlorethamine, chlorambucil, melphalan, dacarbazine, temozolomide, cisplatin, carboplatin, oxaliplatin, nedaplatin, triplatin tetranitrate, phenanthriplatin, picoplatin, satraplatin, carmustine, lomustine, semustine, bendamustine, nimustine, ranimustine, uramustine, busulfan, chlormethine, fotemustine, ifosfamide, streptozocin, bleomycin, mitomycin, altretamine, irinotecan, topotecan, camptothecin, lamellarin, etoposide, teniposide, doxorubicin, daunorubicin, epirubicin, idarubicin, valrubicin, tafluposide mitoxantrone, and amsacrine; and pharmaceutically acceptable salts thereof.

58. A method of inhibiting cancer cell proliferation in a subject in need thereof, the method comprising contacting the cancer cell with
(a) a compound of Formula (I), 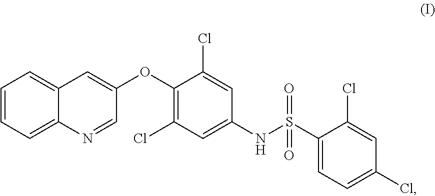 [SMILES: O=S(=O)(Nc1cc(Cl)c(Oc2cnc3ccccc3c2)c(Cl)c1)c1ccc(Cl)cc1Cl]
or a pharmaceutically acceptable salt thereof, and
(b) at least one DNA damaging agent, or a pharmaceutically acceptable salt thereof.

59. The method of claim 58, wherein the at least one DNA damaging agent, or a pharmaceutically acceptable salt thereof, is a single DNA damaging agent selected from cyclophosphamide, mechlorethamine, chlorambucil, melphalan, dacarbazine, temozolomide, cisplatin, carboplatin, oxaliplatin, nedaplatin, triplatin tetranitrate, phenanthriplatin, picoplatin, satraplatin, carmustine, lomustine, semustine, bendamustine, nimustine, ranimustine, uramustine, busulfan, chlormethine, fotemustine, ifosfamide, streptozocin, bleomycin, mitomycin, altretamine, irinotecan, topotecan, camptothecin, lamellarin, etoposide, teniposide, doxorubicin, daunorubicin, epirubicin, idarubicin, valrubicin, tafluposide mitoxantrone, and amsacrine; and pharmaceutically acceptable salts thereof.
